(19) United States
(12) Patent Application Publication
(10) Pub. No.: US 2024/0156368 A1
(43) Pub. Date: May 16, 2024

(54) Wireless Sensors for the Assessment of Cardiac Function

(71) Applicant: Reuven M. Lewinsky, Alonei Aba (IL)

(72) Inventors: Reuven M. Lewinsky, Alonei Aba (IL); Yiftach D. Lewinsky, Alonei Aba (IL)

(21) Appl. No.: 18/388,011

(22) Filed: Nov. 8, 2023

Related U.S. Application Data
(60) Provisional application No. 63/425,641, filed on Nov. 15, 2022.
(60) Provisional application No. 63/463,593, filed on May 3, 2023.

(51) Int. Cl. A61B 5/107; A61B 5/00; A61B 5/0215; A61B 34/20

(52) U.S. Cl. CPC A61B 5/1073; A61B 5/0022; A61B 5/0031; A61B 5/0215; A61B 34/20; A61B 2034/2051; A61B 2034/2063

(57) ABSTRACT

The invention describes a method for continuous or intermittent wireless monitoring of the volume of cardiac chambers, with the ability to combine these with pressure measurements for optimal remote monitoring of cardiac patients. The invention comprises a set of wireless sensors such as resonators or reflectors placed within a cardiac chamber (i.e., the left ventricle), means for sensing the pressure within one or more cardiac chamber, and an external device adapted to interrogate the wireless sensors, determine their relative and/or absolute positions with respect to each other and/or the external device, and calculating the resulting volume of the cardiac chamber and creating pressure-volume loops.

The present invention relates generally to the field of diagnostic medicine, in particular to intrabody measurements using implantable medical devices such as wireless sensors. The present invention claims priority from U.S. provisional applications 63/425,641 and 63/463,593, both of which are incorporated by reference herein.

### FIELD OF THE INVENTION

### Background of the Invention

Remote monitoring of patients with heart disease, especially those suffering from Heart Failure is very challenging. There are several methods for observation of such patients including monitoring of symptoms, weight gain, changes in the ECG, thoracic impedance, and other parameters. These, however, are of limited value due to suboptimal performance (for example in terms of sensitivity, specificity, and rapidity).

Currently, there is no optimal device/method to detect deterioration at its initial phase, when such detection could prevent further deterioration, hospitalization and even death.

Recently, several devices have been introduced into clinical practice which enable the measurement of Left Atrial Pressure (LAP), an increase of which is considered to be a predictor of deterioration of CHF. This is achieved by deploying a pressure sensor in the Pulmonary Artery (indirect measurement, as for example implemented in the Abbott CardioMEMS) or in the Left Atrium (see e.g., the Vectorious V-LAP) which measures and wirelessly transmits the results. These devices show some advantages over past methods but their performance is still suboptimal, relying on the measurement of a single parameter which is often not indicative of a specific person's condition.

The gold standard in investigational cardiology for the assessment of myocardial function is the measurement of the volume of cardiac chambers throughout the cardiac cycle (systole and diastole) and correlating them with simultaneous pressure measurements yielding Pressure-Volume Loops (PVL) where each cardiac cycle is represented by a single loop¹. The analysis of these loops, either single or a sequential series of several cardiac cycles provides valuable information regarding important parameters of cardiac function such as myocardial contractility during systole (inotropy), myocardial relaxation during diastole (lusitropy), preload and afterload. This is currently only possible in experimental models or by using a conductance catheter to yield continuous volume measurements, an invasive method not applicable for routine medical practice^{2 3}. Cardiac chamber volumes can also be assessed by different modalities such as echocardiography, CT or MRI. These however, are only available in hospitals or clinics, require professional staff and are, therefore, not suitable for remote ambulatory monitoring. ¹Sagawa, The End-systolic Pressure-Volume Relation of the Ventricle: Definition, Modifications, and Clinical Use, Circulation 63:6 pp. 1223-1227. 1981²Baan et al, Continuous measurement of left ventricular volume in animals and humans by conductance catheter, Circulation 70:5, pp 812-823 1984³Burkhoff, Pressure-Volume Loops In Clinical Research, J. American College of Cardiology 62:13 pp 1173-1176 2013

Echocardiographic measurements of cardiac chamber volumes are also known to be prone to inter-observer variability and even serial measurements taken by the same operator may yield varying results since it is difficult to assure exact repeatability of the plane or location in which they are taken.

Recently, methods for automatic processing of echocardiographic images are being used to measure cardiac chamber dimensions. These include among others the speckle tracking technology or the enhancement of analysis by applying artificial intelligence (AI) algorithms (U.S. Ser. No. 10/078,893 “Automatic Left Ventricular Function Evaluation” to Guterman et al.). These methods, however, still require the acquisition of high-quality echocardiographic images, a task which requires trained physicians or technicians thus making them unsuitable for home monitoring of cardiac patients. Furthermore, a large proportion of heart failure patients, have a preserved ejection fraction (EF). These cases which commonly result from myocardial diastolic dysfunction are termed ‘heart failure with preserved ejection fraction’ (HFpEF) and as such, their condition is often not accurately assessed by relying on the detection of cardiac chamber contours and measurement of volumes alone without correlating with simultaneous pressure measurements. The current practice for the diagnosis of HFpEF mostly relies on measurements of flow across the mitral valve and mitral annular motion velocity performed by Doppler echocardiography. These, however, are subject to operator skills, suffer from high inter-observer variability and are influenced by various other parameters. A consistent, reliable method for the diagnosis of HFpEF is not yet available for routine medical use.

Currently, there is no device or method suitable for reliable, user-friendly acquisition of cardiac pressure-volume loops, considered to be the gold standard for the assessment of ventricular function, in routine cardiology practice.

### SUMMARY OF THE INVENTION

The invention describes a method for continuous or intermittent wireless monitoring of bodily parameters such as the volume of cardiac chambers. As such, it can provide important information regarding myocardial as well as heart valve function. When combined with pressure measurements, optimal remote monitoring of cardiac patients can be achieved. Thus, the invention provides a wireless sensing solution to monitoring of cardiac chamber volumes as well as PVL, effectively providing the most important parameters of cardiac function, without the need to acquire and analyze echocardiographic images by trained medical professionals. The method can be customized according to each patient's specific medical condition.

The invention is designed as a foolproof device to be operated by the patient, a family member or any other non-professional, which enables remote monitoring of CHF patients, by wireless measurement of the volume of and pressure in cardiac chambers to create Pressure-Volume Loops which provide the best estimate for cardiac function, in a continuous or intermittent manner.

The main components of a primary embodiment of the invention include:
A set of wireless position tracking sensors (these preferably being passive reflectors or resonators adapted for use with ultrasound signals). These are implanted in the body at locations that are prone to relative movement, for instance at various points on the wall of a cardiac chamber. The location of the sensors can be continuously tracked and the distance between them calculated. This enables (for instance) the determination of the volume of the cardiac chamber (i.e., the left ventricle).
Means for sensing and transmitting the pressure within one or more cardiac chamber, again ideally using wireless means.
An external device adapted to track the wireless position sensors, to determine their relative and/or absolute positions with respect to each other and/or the external device, as well as to take readings from the pressure sensor(s) and generate a series of PVLs of the cardiac chamber for further analysis. The external device will generally comprise one or more ultrasound transducers, possibly in the form of linear or matrix arrays.

As mentioned, a preferred embodiment for the position tracking sensors is to use passive reflectors for use with ultrasound signals. Another approach is to use resonant bodies, with resonance occurring at a particular ultrasound frequency. In either case, the external device sends an initial pulse that is either reflected to some degree, and/or causes resonance which yields a reflected resonant signal with a significant amplitude at a specific frequency relative to background reflections. The resulting signal is detected by the external device, which can then determine the position of the sensor using various means of triangulation.

The pressure sensing may be accomplished for instance by means of further sensors, either as stand-alone sensors or alternatively integrated with one or more of the position tracking sensors of the invention, while a third alternative is that pressure is not measured and the volume alone is tracked. It is within provision of the invention to use the resonant body mentioned above for pressure sensing—for instance, by using a body whose resonant frequency varies with external pressure in a predictable and repeatable way.

The invention is primarily intended for continuous or intermittent monitoring with emphasis on outpatient environment. This is useful amongst others for patients suffering from heart failure, valvular heart disease, intracardiac shunts, etc.

Placement of the position sensors assures that serial measurements over time are always made at the exact same location or plane which assures reliability and repeatability of measurements. This will enable the device to compare the results of each examination to those of previous examinations thus providing the ability to perform reliable trend analysis where each patient is his own control and not just determine if the results fit into commonly accepted population-based normal values. As such, the current disclosure may also be integrated into existing or future echocardiography devices used in clinics and hospitals to improve the diagnostic accuracy of routine echocardiographic examinations.

The foregoing embodiments of the invention have been described and illustrated in conjunction with systems and methods thereof, which are meant to be merely illustrative, and not limiting. Furthermore, just as every particular reference may embody particular methods/systems, yet not require such, ultimately such teaching is meant for all expressions notwithstanding the use of particular embodiments.

### BRIEF DESCRIPTION OF THE DRAWINGS

Embodiments and features of the present invention are described herein in conjunction with the following drawings:

FIG. 1 shows a block diagram of system components.

FIG. 2 shows a flowchart of one embodiment of system operation.

FIG. 3 shows a schematic of sensors in place in the heart.

FIGS. 4A, B, C and D show several possible embodiments of the external unit of device.

FIG. 5 (prior art) shows a typical pressure-volume loop (PVL) of the left ventricle.

FIG. 6A shows a possible schematic representation of a multi-layer position sensor and its associated ‘bar-code’ FIG. 6B.

FIGS. 7A, B illustrate examples of pressure-sensitive resonant devices.

FIG. 8A shows resonance curves for various pressures, and FIG. 8B shows a representative graph of resonance frequency vs. pressure.

FIG. 9 shows the storage and analysis of pressure-volume results on the local external device as well as on a cloud-based database.

### DETAILED DESCRIPTION OF PREFERRED EMBODIMENTS

The present invention will be understood from the following detailed description of preferred embodiments, which are meant to be descriptive yet not limiting. For the sake of brevity, some well-known features, methods, systems, procedures, components, circuits, and so on, are not described in detail.

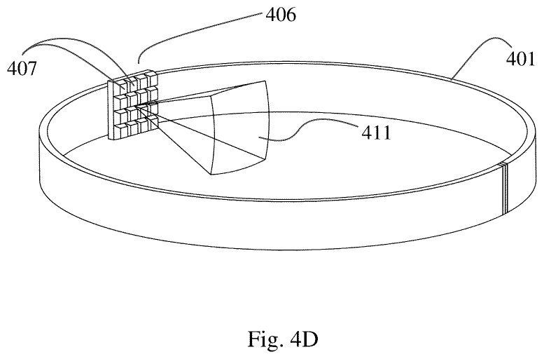

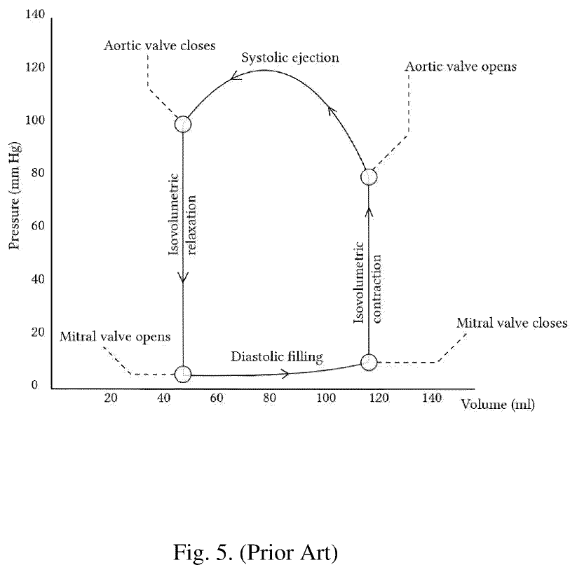

The ‘gold standard’ for assessing myocardial function and other critical parameters of cardiac function is the pressure-volume relationship for the left ventricle, this relationship being reflected in the ‘PVL’ or pressure-volume loop for left ventricle. For purposes of determining ventricular volume, either invasive means such as catheters are used, or ultrasound imaging and associated image processing have hitherto been employed. Invasive means have obvious drawbacks, while ultrasound with volume determination requires the acquisition and analysis of high-quality images by experts in Echocardiography, and even these are prone to interobserver variability. A representative PVL is shown in FIG. 5.

The current invention describes a method for continuous or intermittent wireless monitoring of the volume of cardiac chambers, with the ability to combine these with pressure measurements for optimal remote monitoring of cardiac patients, without the need to acquire or analyze high quality ultrasound images.

Monitoring of various bodily areas has been introduced for example in U.S. Pat. No. 6,498,944 “Intrabody Measurement” to Ben Haim, which discloses a catheter-based method of measuring the size of a body area using a set of position sensors mounted on the tips of one or more catheters, and calculation of distances between them. The position sensors comprise miniature coils which reflect electromagnetic waves, which are produced and detected by suitable apparatus outside the body.

Similarly, U.S. Ser. No. 10/918,858 “Cardiac volume sensing via an implantable medical device in support of cardiac resynchronization therapy” discloses an implantable medical device having two electrodes and coupled to them, configured to identify a measure of impedance between the two electrodes.

Due to the obvious drawbacks of either using an invasive means such as a catheter or an active electrical measurement, it is an object of the current invention to introduce a method that avoids both the use of catheters and active impedance measurement for determination of time-varying bodily volumes.

The main components of a primary embodiment of the invention include:
A set of wireless sensors deployed in a cardiac chamber. These are preferably passive devices adapted to have high ultrasound reflectivity or a specific resonant frequency. Using true range multilateration, triangulation or similar means, or the spatial information which can be derived from the ultrasound transducer per its specific design, the location of the sensors can be continuously tracked and the distance between them calculated, to enable determination of the volume of the cardiac chamber (i.e., the left ventricle). The wireless sensors may be passive reflectors which can be tracked by use of ultrasound signals.
Means for sensing the pressure within one or more cardiac chamber.
An external device adapted to track the wireless sensors, to determine their relative and/or absolute positions with respect to each other and/or the external device. The external device will generally comprise one or more ultrasound transducers, possibly in the form of linear arrays, phased arrays or matrix arrays.

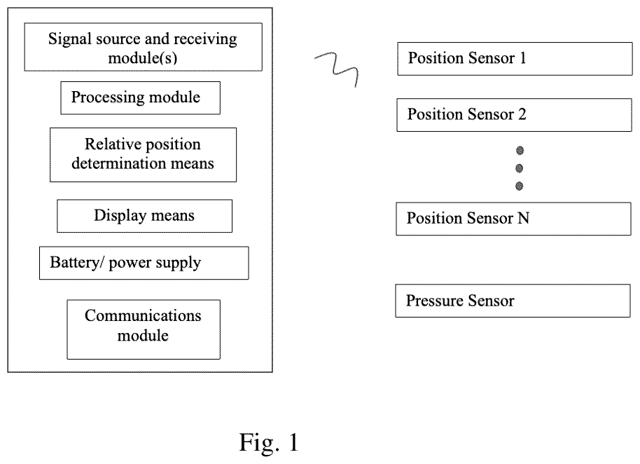

A block diagram of one embodiment of system components of a wearable external unit is shown in FIG. 1. On the left an external unit having a signal source and receiving module, preferably one or more ultrasound transducer elements, communication means, processing module, relative position determination means, display means and a power supply to power all these components is shown. The implanted sensors of the system are shown on the right, including two or more position sensors and at least one pressure sensor. As mentioned above, it may be the case that a pressure sensor is not needed which may apply in the case of using the invention for certain clinical indications.

The communication means of the external unit may include modules intended for communicating with other components of the system as well as with other devices such as PCs, smartphones, etc.

The results of each examination (namely, parameters derived from pressure-volume data) may be stored on the external device for comparison with previous or following examinations. This will enable physicians or other users to perform trend analysis to determine if the patient's cardiac health has remained stable, has deteriorated or improved.

The display means of the external device will enable operation of the device as well as provide user feedback regarding signal quality, analysis of results, etc.

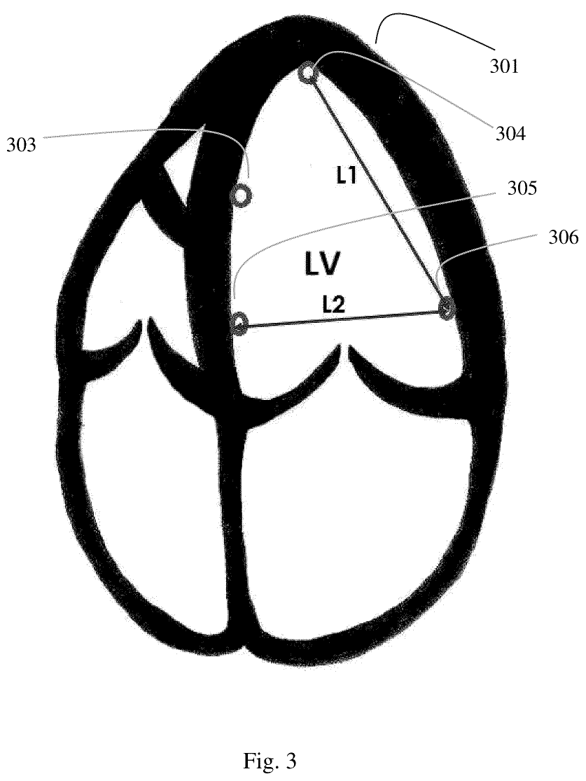

An example of an embodiment of the implanted part of the invention with one pressure sensor and three position sensors is shown in FIG. 3, with the left ventricle 301 being supplied with position sensors 302a,b,c and a pressure sensor 303.

### Position Sensors

The position sensors are tracked by an external means (in a preferred embodiment this being by use of ultrasound signals from passive sensors, but also possibly being implemented by electromagnetic sensors or other means) to determine their relative displacement Δx and absolute distance.

### Reflectors

For embodiments using passive ultrasound reflectors, the shape, size, design (solid or hollow), the surface features and material of the reflectors will have an effect on the nature and intensity of the reflections therefrom. The difference between the acoustic impedance of the reflector and that of the surrounding medium will determine the intensity of reflection or ‘reflection coefficient’. The acoustic impedances of several materials are shown in the following table, along with the reflection coefficient expected at an interface between blood and the material in question; thus, the reflections expected from the materials in the table are all near 80% or above, and depending on their biocompatibility may be good candidates for reflectors. Non-biocompatible materials will be coated by or encapsulated within a biocompatible material. Such materials may include among others polyurethane, ceramic, medical grade silicon, titanium, Nitinol, etc.

TABLE 1
Reflection Coefficients for several materials, in blood

| | Acoustic Impedance | |
|---|---|---|
| Material | [10\hat{ }6 kg/m\hat{ }2 s] | Reflection coefficient |
| Air | 0.0004 | 0.999 |
| Alumina | 9.7 | 0.51 |
| Blood | 1.6 | 0 |
| Bone | 4.7 | .24 |
| Copper | 33.4 | .83 |
| Glass | 11.6 | .57 |
| Silver | 37.7 | .84 |
| Steel | 40.3 | .85 |
| Titanium | 27.6 | .79 |
| Tungsten | 97.8 | .93 |
| Zirconia | 42 | .86 |

It is within provision of the invention to use any material for the reflectors, formed into any shape; however, as the reflection will depend upon the orientation of the reflectors (which may change with heart wall movement, and cannot be controlled) it may be found useful to use regular polyhedra, cylinders, ‘corner cubes’, or spheres for the reflectors. The corner cube has the useful property that the reflected wave is entirely in the direction of the incoming wave. Whatever its form, the reflector may be hollow and preferably filled with air, to take advantage of further nearly-perfect reflections of the transmitted wave off of the interior walls of the reflector.

In the preferred embodiment, the position sensors can be any kind of passive reflector and/or resonator adapted to return and/or resonate at any predetermined specific wavelength, preferably of ultrasound.

A smooth, hollow titanium or nitinol sphere of several wavelengths diameter is an example of a simple, biocompatible isotropic specular reflector. As medical ultrasound systems often use a 3.5 MHz frequency with a wavelength of 0.44 mm, this dictates use of a sphere of about 1 mm diameter or larger.

In another possible embodiment, the passive reflectors can be made of a porous material containing air or fluid bubbles or pores with a size which can range from tens to hundreds of micrometers (μm). One such material which may be used for the reflectors is Hydroxyapatite, a biocompatible material already used in various medical implants, mainly in the fields of orthopedics and dentistry which can be manufactured with various degrees of density and porosity. Hydroxyapatite may be categorized under the family of ceramic materials and is just one example of this group of materials. There are also metallic biocompatible materials which may be produced with varying degrees of porosity such as Nitinol and Stainless Steel. Similarly, various biocompatible polymers polyethylene, polyurethane, and poly (lactic-co-glycolic acid) (PLGA) can also be used to manufacture the position sensors.

A porous material has a unique “ultrasound signature” depending among other factors on the size and distribution of pores. Such a material includes multiple air (or fluid) to solid interfaces, each of them with a specific reflection, absorption and scattering pattern. The presence of multiple interfaces between the solid matrix and the pores can lead to multiple reflections of ultrasound waves, creating complex patterns within the material such that the overall reflected signal from a porous reflector can be distinguished from reflections generated by surrounding tissue.

As will be appreciated by one skilled in the art, the reflectors used for purposes of the invention may also comprise resonant devices having one or more resonances that may be easily picked up by appropriate signal processing methods employed by the external interrogation device. It is within provision of the invention that the resonances of the reflectors used for position detection be made distinguishable, for instance by use of the hollow spheres mentioned above, each having a different outer and/or inner radius; use of solid spheres of different radii, porous materials with different porosities, pore sizes and void materials; or objects with different resonance properties such as 2- or 3-D arrays of hemispheres. As a simpler alternative to large arrays, several coupled position sensors may be arranged in a known pattern to produce unique return signals. Since the resonances of the objects being used may be well characterized before use, the process of detecting their reflections and triangulating their positions is made somewhat simpler.

### Resonant Devices

Another embodiment for the position sensors involves resonance. Resonant devices also use the acoustic (ultrasound) waves emitted from the external transducer towards the sensor element. In this case however, instead of simply reflecting some part of the incoming signal, the sensor element enters into a resonating state upon absorption of an incoming signal near the frequency of the element. The specific natural frequency of each sensor results from its physical characteristics (e.g., stiffness, mass and geometry) allowing each sensor to have a unique signature which can be detected by analyzing the resultant (reflected) signal. The external acoustic waves are ideally emitted at such frequency which will yield an optimal amplification versus attenuation ratio to provide the best SNR. A resonant object will easily vibrate at any of its resonant frequencies, and vibrate less strongly at other frequencies. It will “pick out” its resonance frequency from a complex excitation, such as an impulse or a wideband noise excitation—in effect, filtering out all frequencies other than its resonance. As will be discussed in the section on pressure sensing below, this resonant frequency may depend upon the external pressure, and this change in resonance may be sensed by the external device of the invention, allowing for position and pressure sensing in a single device.

### OTHER EMBODIMENTS

In another embodiment, the sensors may be active, for example receiving power from an external source such as radiofrequency or ultrasound and transmitting data to an external receiver by means of RF or any other applicable wireless transmission method, or being supplied with internal power means such as batteries or energy harvesting means. The sensor can be made of any material adapted to interfere with, reflect, or transmit any specific wavelength or set of wavelengths of sound, ultrasound, electric field, magnetic field, or other type of energy.

In yet another embodiment, the sensors may be piezoelectric sonomicrometry crystals receiving power from an external source such as radiofrequency or ultrasound, emitting ultrasound signals to, and receiving ultrasound signals from other piezoelectric sonomicrometry crystals implanted in different locations within the cardiac chamber, and transmitting data to an external device by wireless means.

The sensors (reflectors) may be in the form of an encapsulated cluster of microbubbles.

In one embodiment of the invention, the sensors may be brought to their target location by a trans-catheter approach, either via the arterial system to the left heart chambers, or the venous system to the right heart chambers. The left heart may also be reached by the venous route through a trans-septal puncture in the right atrium towards the left atrium.

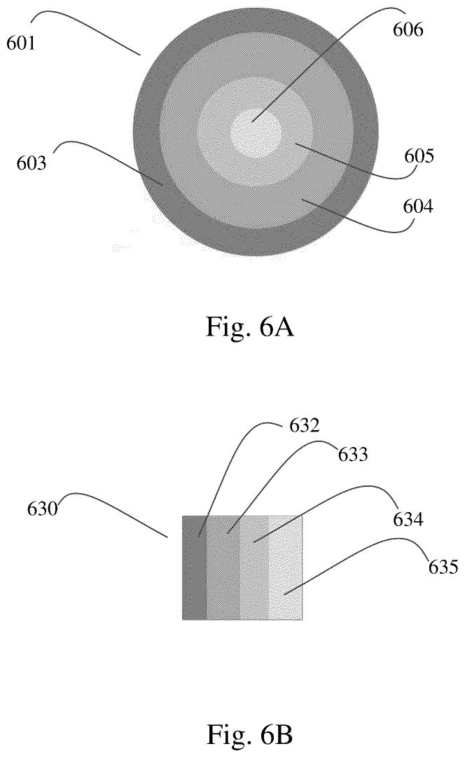

Another example is illustrated in FIGS. 6A and 6B. Here the position sensor (601) includes several layers (603-606) each with a specific thickness and acoustic impedance. This will cause the reflected ultrasound beam to travel back from each interface between layers with an extra time delay relative to the layer thickness, while the intensity of the signal will be proportional to the reflection coefficient at the interface between the layers. Such a design will produce reflectors with specific “signatures” allowing the system to use a multitude of reflectors (601) while enabling to distinguish between them as they are being tracked. The reflector-specific signature can be presented as a “barcode” (630) in which the width of each bar (632-635) is relative to the thickness of the layer 603-606) while the color or grayscale is relative to the acoustic impedance of each layer. The bars 632-635 can be arranged in an inside-out direction or vice versa. The barcode (630) presentation is not required for the practical implementation but may serve other uses of the system, e.g., the user may scan a barcode (630) of a certain reflector (601) and ask the system to locate only this specific reflector (601).

One useful aspect of this embodiment is that certain functions of the returned signal will remain invariant to the angle between interrogating signal and reflector. For example, the ratios between time delays from the different interfaces will remain the same, while the values alone will change with angle in. predictable way. Thus, the identity of a particular reflector will remain easy to determine (for example by use of the aforementioned invariant ratios), while the angle at which it is disposed relative to the interrogator can be calculated from the values themselves.

It is expected that most sensor implantation procedures will be performed as an additional step in a cardiac catheterization performed for other purposes such as coronary angiography, valve repair or replacement, or the like. Alternatively, the sensor placement may also be a stand-alone procedure. Another method for deploying the sensors at their target location may employ an injection mechanism whereby the sensors are embedded in the myocardium and instantaneously covered by the surrounding tissue such that further fixation may not be required.

The sensors can be deployed at their target on the wall of a cardiac chamber by several methods including but not limited to attachment by a hook-like or spring like mechanism, self-expanding Nitinol anchors, or other means as will be clear to one skilled in the art. It is expected that the sensors will undergo endothelialization within a short period such that they will not be exposed to the blood in the heart thus eliminating the need for anticoagulation. The functionality of the passive reflectors will, however, not be affected by being covered by endothelium or embedded in the myocardium. Anchoring of the sensors to the myocardium is not only a safety feature. It assures that serial measurements over time are always made at the exact same location or plane which assures reliability and repeatability of measurements.

As discussed above, the sensors will be either made of, or covered by a biocompatible material. The material to be used has to be non-degradable to ensure lifetime endurance.

### External Unit and Ultrasound Probe

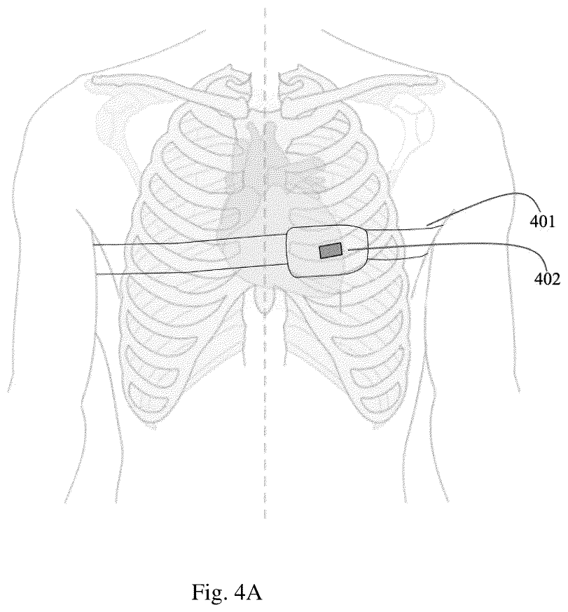

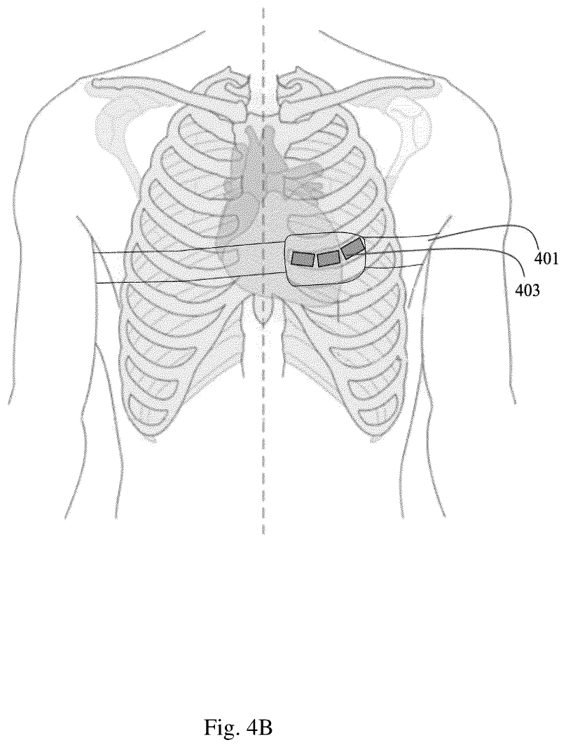

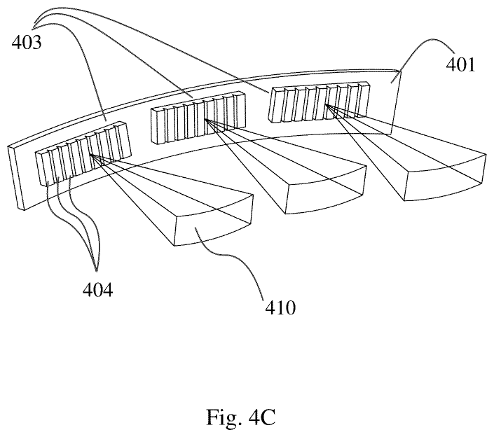

The ultrasound transducers are configured to act as a “Medical Radar” or data transmission system. To be able to determine the exact position of each sensor, the method requires a way of achieving true range multilateration or triangulation (similar to applications such as surveying, navigation, etc.). This can be achieved by either using a series of existing ultrasound transducers (i.e., a phased array) or by creating a series of custom-made arrays of ultrasound emitting and receiving elements angled relative to each other, either within a single probe or on a strip/belt etc., such that angulation is enabled. Such an implementation is shown in FIG. 4 where the phased arrays are placed on a band around the chest, where they may be positioned directly over the heart such that the signals are clear. An alternative embodiment of the method can be done by using a matrix ultrasound probe which scans at two planes perpendicular to each other and extracting spatial information from the transducer such that true range multilateration or triangulation may not be required.

An embodiment using a chest strap bearing an ultrasound probe is shown in FIG. 4A, where a belt or strap 401 is provided with a matrix ultrasound probe 402 that employs a set of transducer elements. Alternatively, a set of several linear (or phased) arrays 403 as shown in FIG. 4B may be used. The linear arrays will generally be angled relative to one another. Each array may be linear 403 (FIG. 4C) or itself of a matrix array form 406 (FIG. 4D). The arrays will generally have sets of transceiver elements (transmitting and receiving) 404, (FIG. 4C) although in some implementations separate elements may be used for transmitting and receiving. In FIG. 4D the transceiver elements 407 are shown in a square matrix. The relatively flat beam pattern 410 of the linear array, scanning a “slice” of an organ is shown in FIG. 4C, while the more volumetric (pyramidal) scanning pattern 411 of the matrix array is shown in FIG. 4D. The arrays and elements may be angled relative to each other. The belt bearing the array or arrays is worn obliquely on the chest over an intercostal space using an “acoustic window” between the ribs allowing the ultrasound signals to reach the heart without being obstructed by bone and cartilage. This approach is resembling the commonly used Apical and Parasternal Long Axis views.

It is within provision of the invention that suprasternal and substernal approaches may be applied using a hand-held transducer, without using such a belt. In the case of a matrix probe or matrix array as shown in FIG. 4D, since the probe scans in two planes perpendicular to each other, a single probe may suffice. It is within provision of the invention to use any of the available acoustic windows, including the aforementioned parasternal and apical windows, as well as the subcostal, suprasternal, and any other windows that may be found effective for practice of the invention.

It is within provision of the invention that various configurations of transducers or sets of transducers (including transceivers or separate transmitters and receivers) may be used to adapt to specific clinical needs.

The external unit may be divided into two or more physical parts, if for instance it is found that putting all necessary electronics, power supply, display means, communications means and so on upon a chest strap as in FIG. 4 is not possible. Thus, the chest strap of FIG. 4 may carry the ultrasound arrays, while a separate power/computing unit in communication with these arrays performs the data analysis, transmission to the cloud, supplies power, and so on. This separate power/computing unit may be in wired or wireless communication with the chest strap transducers.

It is within provision of the invention that the ultrasound elements include separate or unitized transmit and receive elements, and that the arrays be adapted for placement in contact with the skin, for example being arranged upon a flexible substrate adapted to conform to the body to promote good contact with the skin. To assure proper coupling of the ultrasound signal to the subject's skin, an encapsulated gel unit may be used between each transducer and the skin. Alternatively, a recess in the element holding the transducers opposite the skin may be filled by coupling gel prior to each examination.

The position and angle of the ultrasound transducers will be determined in a way which will ensure the acquisition of good quality signals from the passive reflectors depending on their specific locations within the heart. Such positions may include among others an intercostal space, substernal or suprasternal or others.

As will be clear to one skilled in the art, an advantage of phased arrays is the steerability of the main beam; this may be used for instance by scanning an area with the beam to simultaneously detect and track at least two reflectors, and then using the angle with the greatest response to achieve a high SNR. The number of elements in each transducer and their sequence of activation will be adapted to ensure optimal performance. Similarly, the delay in activation of elements will be optimized to minimize interference between their signals. When implemented by an array of arrays, the delay between activation of arrays can be modulated to achieve optimal performance and avoid signal interference.

Also, as known to one skilled in the art, in order to determine the location of a moving object using multiple ranges (distances) between the moving object and multiple spatially separated known locations. In an ultrasound probe, each transceiver element is an independent reference. The combination of several ultrasound probes, each consisting of multiple transceiver elements arranged in either a linear (or phased array) or a single matrix probe provides a plurality of measurements taken from different angles assuring a high accuracy and spatial resolution.

As mentioned, there is also provided a method and apparatus for determining the distance of two or more sensors from each other. The invention may also be applied to continuous or intermittent tracking of a plurality of sensors and determination of their relative position.

Specifically, the invention allows for measurement of the distance between two sensors or more within a cardiac chamber (i.e., the left ventricle). The position of each sensor relative to the external device is made by using true range multilateration or triangulation or alternatively the absolute positions of the sensors may be determined and then the distance calculated by simple geometric means.

When using a matrix ultrasound probe, the inherent properties of such a transducer, which scans a targeted volume in two planes perpendicular to each other, may enable the calculation of distances between sensors possible without traditional true range multilateration or triangulation methods.

Magnetic field means may also be used (instead of or in addition to ultrasound means) for relative or absolute position sensing of the wireless sensors. In this case the implanted sensors would have a specific magnetic structure adapted for remote detection.

The sensors can also be deployed in more than one cardiac chamber (i.e., left atrium and left ventricle, left and right ventricles, etc.) For some applications, the sensors can be deployed at locations not within cardiac chambers but adjacent to them (i.e., the coronary sinus, aorta, pulmonary arteries or veins, inferior or superior vena cava). The sensors may be integrated into any existing or future implanted cardiac device such as artificial valves, PFO occluders, pacemakers, implantable cardioverter-defibrillator (ICD), left ventricular assist devices (LVAD), etc. The sensors are adapted with specific geometry, material, surface (smooth—rough) or echogenic coating, with an engineered response to specific energies/signals to create a specific reflection footprint ranging from “stealth” to noisy (these corresponding to non- or poorly-reflective, to highly-reflective, respectively).

The measurements can be taken sporadically or in a continuous, real-time manner. Assuming a heart rate of 60-120 bpm (cardiac cycle of 0.5-1.0 sec), a sample frequency of 100 Hz may be used to achieve the desired temporal resolution, especially important not to miss the exact points of deflection of the PVL curve)

In the preferred embodiment wherein passive ultrasound reflectors are implanted in the heart to determine volume, the external device will comprise an ultrasound transducer. For echocardiography, most transducers work at a frequency of 2-4 MHz allowing penetration to the required depth within the chest.

While the external device described in the invention may be custom made when applied for remote monitoring, the high quality, operator-independent measurements provided by the technology and based on the invention, may be used in the hospital or clinic setting where the technology could be integrated into existing or future ultrasound devices and transducers. This will provide clinicians with a reliable, consistent way for assessing the existence and severity of heart failure, especially in the subset of patients suffering from diastolic dysfunction or HFpEF.

It is within provision of the invention that the external device scans a certain “slice” of the organ when using a linear (or phased) array, while a matrix arrangement of piezoelectric elements allows their phasic firing to produce an ultrasound beam that can be steered in vertical (axial), lateral (azimuthal) and antero-posterior (elevation) directions in order to acquire a volumetric (pyramidal) data set. These scanning properties assure that at least two sensors are identified within that slice or volume and tracked for at least a few cardiac cycles at each session.

It is within provision of the invention that the external device provides the user with feedback assuring that the signal reflected from at least two sensors is of sufficient quality to enable reliable results. This may be accomplished by various self-check means such as thresholding on values of pressure and volume, the area of the PVL, time derivatives of volume and/or pressure, signal strength, signal-to-noise ratio, and so on.

Unlike other imaging modalities, there is no need to acquire images for producing the desired results. Images may, however, be displayed as a side product of the system as part of the feedback provided to the user, such as highlighting the identified position sensors within the overall ultrasonographic scan.

As described above, the placement of the position sensors assures that serial measurements over time are always made at the exact same location or plane which assures reliability and repeatability of measurements. This will enable the device to compare the results of each examination to those of previous examinations thus providing the ability to perform reliable trend analysis where each patient is his own control and not just determine if the results fit into commonly accepted population-based normal values.

The external device performs certain calculations on the measurements, including deriving cardiac chamber volume from one or more distance measurements, and calculating the pressure-volume loops as they accumulate over time, and functions of this loop such as End-Systolic pressure Volume Relationship (ESPVR), End-Systolic Elastance (EEs)—the slope of ESPVR considered to be the best indicator of myocardial contractility, End-Diastolic Pressure Volume Relationship (EDPVR) Arterial Elastance (Ea)—a measure of afterload, and the like.

The results calculated by the external device may be stored, transmitted and further analyzed by cellular phones, cloud, other computers, etc.

While the invention is primarily about deriving parameters from ventricular pressure-volume data, a plurality of clinically significant parameters can be derived from ventricular volume alone. These include among others Ejection Fraction, End-diastolic Volume, Stroke Volume, Cardiac Output, Cardiac Index and Global Longitudinal Shortening (GLS).

An example of an embodiment of the implanted part of the disclosure with one pressure sensor and two position sensors is shown in FIG. 3, with the left ventricle 301 being supplied with position sensors 304, 305, 306 and a pressure sensor 303.

Further shown in FIG. 3, is an example of specific locations of the position sensors within the Left Ventricle. The figure shows the heart as seen in the Apical 4 Chamber view, one of the commonly used views during echocardiography. The distance between the position sensor deployed at the endocardial aspect of the apex of the ventricle and the position sensor deployed at the endocardial aspect of the lateral ventricular wall (L1) represents the longitudinal or axial dimension of the left ventricle while the distance between the two position sensors deployed at opposite locations on a cross-sectional plane at the sub-mitral level of the base of the ventricle of the ventricle (L2) represents the horizontal dimensions of the ventricle.

Based on the assumptions that the left Ventricle resembles an ellipsoid shape, and that the distance L2 is similar to the distance perpendicular to it between the endocardial aspects of the ventricular wall at the same level, and taking simultaneous measurements of the 2 distances (L1 and L2) enables calculation of the Left Ventricular Volume according to the formula:

$$LV Volume = \frac{π}{6} \times L 1 \times L {2}^{2}$$

While the formula of calculating the volume of an ellipsoid to estimate ventricular volume is well established in the cardiology practice, other formulas may be used for the calculation of ventricular volume from the distances between the position sensors. These may be derived from actual measurements of ventricular volumes by imaging modalities such as CT, MRI and Echocardiography by means of non-linear regression or other mathematical methods. Adaptive formulas for ventricular volume calculation may, however, be used depending on the exact location in which the position sensors will be deployed as well as on data accumulated on specific patients as well as on patient groups and populations.

In addition to estimation of ventricular volume as described above, the change in the distance L1 between diastole and systole will enable to measure Global Longitudinal Shortening (GLS), a parameter which is gaining a growing interest as an indicator of myocardial function.

### Pressure Sensing Means

As mentioned, the measurements of the device can be correlated with measurements of other parameters such as pressure within one or more cardiac chambers. The pressure sensing may be accomplished by means of separate wireless sensors, or alternatively integrated with the volume sensing means. A third alternative is that pressure is not measured and the volume alone is tracked.

Pressure sensing may be accomplished by one of several means as will be familiar to one skilled in the art. For example, an active (powered) MEMS pressure sensor may be used, for example being powered by means of an external power source, such as an RF or other wireless power source.

A second option is to use a resonant sensor, this being any device that has the capability of resonating at a defined resonance frequency upon excitation by energy/power from an external source, preferably ultrasound. This resonance may be changed as a result of a change of a physical variable such as pressure to which it is exposed, for instance by means of geometric changes to the device that result from pressure changes. As will be appreciated, the resonant sensor may in this case be entirely passive, a significant advantage in this application. This implementation has the advantage that it allows position sensing and pressure sensing to be combined into a single device—the resonant sensor is used both for position sensing (by triangulation) and pressure sensing (by sensing a pressure-dependent shift in resonant frequency).

For the purpose of this application, a resonant sensor is meant to encompass any device with a certain size, shape, stiffness and elasticity which result in a defined natural resonance frequency and has the capability of resonating at this defined frequency upon excitation by an external energy source, i.e., ultrasound. In addition, the device has the capability of changing its resonance frequency in a defined way as a result of a change of a physical variable, such as pressure that the device is being exposed to which affects the device's shape (for example, causing deformation), density or stiffness.

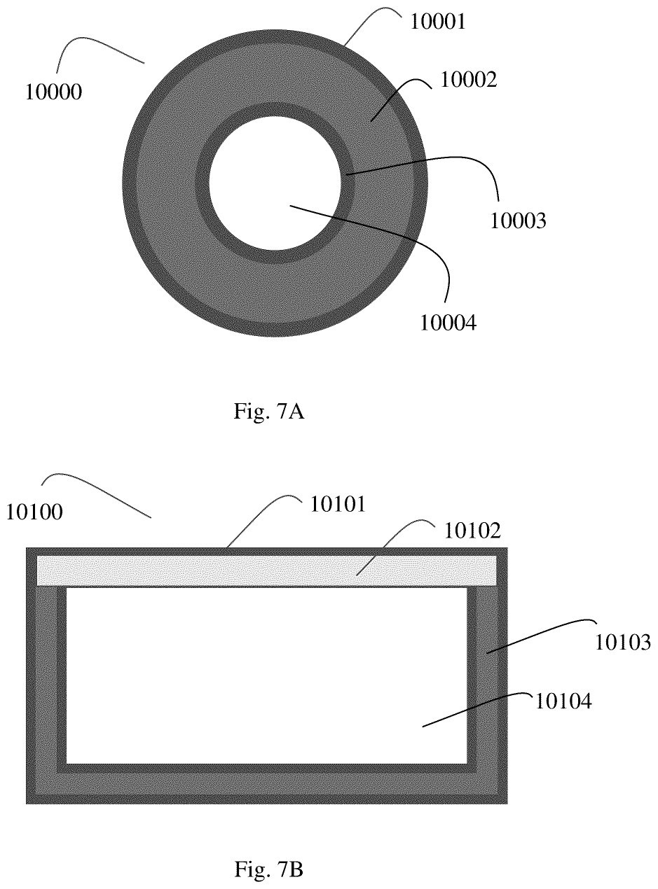

To take some simple examples, spherical or rectangular devices of FIGS. 7A, B represent devices that may be used as resonant bodies that allow for both position measurement by means of triangulation, and pressure measurement by means of changes in resonant frequency with pressure. FIG. 7A shows a sphere 10000 having an outer biocompatible coating 10001, a body made of material 10002, inner coating 10003, and inner volume 10004. The inner volume may be for instance air at atmospheric pressure, allowing for high ultrasound reflectivity and high dependence upon external pressure. Alternatively, the sphere may be solid, having only an outer biocompatible coating 10001, or it may itself be made of some biocompatible material thus obviating the need for a coating. In any case, the makeup of the sphere is designed such that the frequency at which the sphere resonates will depend on the external pressure, allowing for such a device to act both as position and pressure sensor. This may be accomplished for instance if the sphere is somewhat compressible, allowing it to compress under higher external pressure and thus resonate at a higher frequency due to its greater stiffness when compressed. FIG. 7B shows another possible embodiment of a rectangular form, where again an outer biocompatible layer 10101 surrounds an object this time of rectangular form, with a top membrane 10102 having characteristics that may differ from that of the ‘box’ material 10103, for example being of greater flexibility and/or being made thinner. Again, an inner volume 10104 may be filled with air or another medium, or the device may be solid and dispense with use of a separate inner volume, and the material of the device itself may be intrinsically biocompatible. For all such devices the desired characteristics are that the device has one or more distinct resonances, which are affected measurably by changes in the pressure of the surrounding medium. Thus, non- or semi-rigid materials such as polymers, including rubbers and plastics, gels, or the like may be found of use to form these objects.

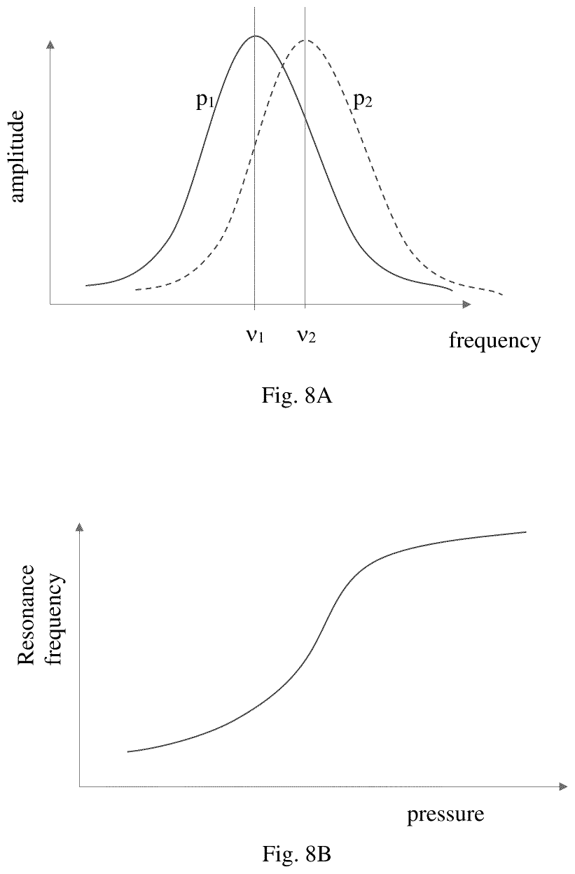

As mentioned, the resonant frequency of the pressure sensing device will ideally shift in response to external pressure change in a predictable way. A representative situation is shown in FIGS. 8A, B; at an initial pressure p₁ (leftmost curve) the resonant frequency of the pressure sensing device may be v₁. The graph shows the amplitude (y-axis) of the reflected signal from the resonating body in response to a driving signal of a given frequency (x-axis), with a peak for resonance at ω₁. If the external pressure changes to p₂ the resonant frequency of the pressure sensing device will likewise shift to v₂. (rightmost curve). The change in resonant frequency as a function of pressure may thus be determined as in FIG. 8B. This curve is assumed to be determined during a calibration step.

The shift in resonance of such a device in response to external pressure changes may be detected in several ways. Perhaps most simply, the incoming signal frequency may be left unchanged, while the peak frequency of the returning signal is analyzed and used to determine pressure. As long as the resonance response curve (e.g., the curve of FIG. 8A) is not too steep, the resonant body will still respond to the off-resonance incoming signal frequency to some extent even if this incoming frequency is not exactly at the resonance frequency. Thus, even for a driving signal of frequency ω₁ the response signal will still respond and show a peak at ω₂.

Another approach is to constantly sweep or otherwise change the incoming signal frequency in an attempt to find the frequency of maximum response, which will occur at the resonant frequency of the pressure sensing body. A third approach is to use a wideband or impulse signal to excite resonance in the resonating body—since a wide band of frequencies are contained in the exciting signal, the resonating body will be driven at resonance to some degree.

A fourth approach is to use Doppler shifts in the echo from the incoming signal; due to vibration of the pressure sensing body there will be regular Doppler shifts in the echo off the pressure sensing body. For this approach it may be found that two incoming signals may be more useful; one being used to excite the resonance in the pressure sensing body, and a second being used to determine Doppler shifts in the echo from the now-vibrating pressure sensing body.

In some embodiments, one of the position sensors may be integrated with a pressure sensor (for example being encapsulated together) such that the total number of sensors can be reduced.

As an example of the utility of the invention, left ventricular volume measured throughout cardiac cycles when correlated with continuous pressure measurements can yield pressure-volume loops indicative of myocardial function.

It is further within provision of the invention that information be derived regarding function or dysfunction of cardiac valves on the left side of the heart (i.e., mitral or aortic stenosis or regurgitation). The same may be applied to valves in the right side of the heart (i.e., Tricuspid or Pulmonic stenosis or regurgitation).

The measurements may also be correlated with or gated with the electrical activity of the heart (ECG).

The system may also integrate measurements derived from one or more miniaturized three-axis (3D) accelerometers. When positioned in the atria, an accelerometer can provide information on dysrhythmias (i.e., atrial fibrillation) which would enable refining of PV loops, while when positioned in a cardiac ventricle, identification of dyskinetic regions can improve the assessment of the displacement between the position sensors and the orientation thereof. Alternatively, acceleration estimates may be made using the position data provided by the position sensors if this is of high enough accuracy.

Measurements may be modulated by external manipulations (brachial or femoral cuffs to increase afterload, Valsalva maneuver or positional change to reduce preload) in order to produce a series of pressure-volume loops displaced relative to each other thus enabling the calculation of end-systolic pressure-volume relationship (ESPVR), end-diastolic pressure-volume relationship (EDPVR) and other parameters of myocardial function.

The invention is primarily intended for continuous or intermittent monitoring with emphasis of outpatient environment of patients suffering from heart failure but also valvular heart disease, intracardiac shunts, etc. However, the high quality, operator-independent measurements provided by the technology and based on the invention, may be used in the hospital or clinic setting where the technology could be integrated into existing or future ultrasound devices and transducers. This will provide clinicians with a reliable, consistent way for assessing the existence and severity of heart failure, especially in the subset of patients suffering from diastolic dysfunction or HFpEF.

It is within provision of the invention that it be applied to any organ in the human body. As an example of further uses, the invention can be used to continuously monitor the excursion of movement of the diaphragm between the chest and the abdominal cavity, an indicator of pulmonary function and other parameters in chronic lung disease, some neurological conditions, and in the critically ill.

The ultrasound transducers to be used in the invention may include wireless handheld transducers, fingertip probes, wearable transducers such as a “belt-like” set of ultrasound transducers (set of arrays), a combination of arrays of transmitters angled relative to each other, ultrasound on a chip, with capacitive micromachined US transducers as an alternative to piezoelectric transducers, or externally powered wireless implanted sonomicrometry crystals.

### Calibration

By means of a calibration, the relation between the distance Δx between the two (or more) sensors and the volume V_{LV} of the left ventricle may be determined. Since the relationship between distance and volume is highly dependent on the position of the sensors within the ventricle and on the individual patient's heart size, morphology and function, a calibration will be obtained once during the procedure in which the sensors are deployed and then relied upon to determine volume V_{LV} from the distance Δx. The calibration may be repeated when the patient undergoes cardiac catheterization for other indications. It further may be the case that the exact shape or coefficient of the calibration may prove to be unimportant, and rather that changes in the pressure-distance relationship over time are of sufficient clinical importance. In this case, the distance-pressure curve may be used as a proxy for the volume-pressure curve.

Furthermore, imaging means such as those employing ultrasound, echocardiography, CT or MRI imaging may be used once after implantation to perform an initial calibration step linking the ultrasound reflections from the position sensors with volume changes.

As an option where after deployment of the position sensors, the physician may once perform a “registration” procedure where he will mark the position sensors on an echocardiographic scan. The now-known coordinates of the position sensors may then be used by the inventive system to our system such that the area where the external unit must search for the implanted sensors will be narrowed significantly. Alternatively, this registration may be used for better placement of the external unit (402 in FIG. 4A).

### Operational Steps

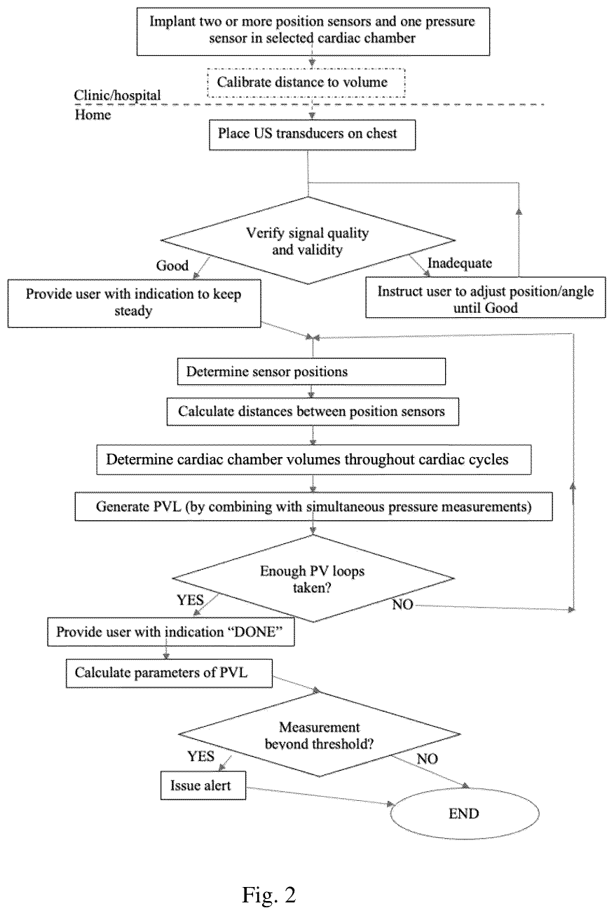

A simplified flowchart of one embodiment of the invention is shown in FIG. 2. First the sensors of the invention are implanted by means that will be described below. Once implanted, the calibration step described above is carried out, if necessary. These steps will generally be performed in a hospital setting. After being discharged, the patient, a family member or any other non-professional, can activate the external device to carry out a repeated sequence of distance and pressure measurements. This is done by moving the ultrasound transducer, or the belt holding an array of arrays of ultrasound emitting and receiving elements until receiving feedback from the external device in the form of a color indicator, sound or other, assuring a good signal quality has been achieved, and directing the patient to maintain this position as stable as possible. Then the device repeatedly measures the distances between position sensors, correlates them with pressure measurements until several pressure-volume loop (PVL) can be generated. When completed, the user receives another feedback “DONE” assuring a satisfactory examination session has been completed and the device may be switched off and removed from the chest. The PVL allows for several values to be calculated including measures of cardiac preload, afterload, myocardial contractility and relaxation. A step of measurement validity is carried out, and if the measurements are valid then the results are stored and may be sent to a network. The results are also compared to predefined thresholds, and if these are exceeded then an alert may be issued. The results may also be compared to the same patient's previous examinations such that deterioration or worsening are detected even if absolute values are still within the normal range. This may take the form of a local alarm as well as an alert sent to medical care providers, phone contacts, and so on. The frequency of measurements may be determined by the caring physician based on the type and severity of the patient's disease, and may range from one or a few measurements per day to one or a few measurements per week to allow for useful predictions of cardiac behavior to be made in time for interventions to be taken if necessary.

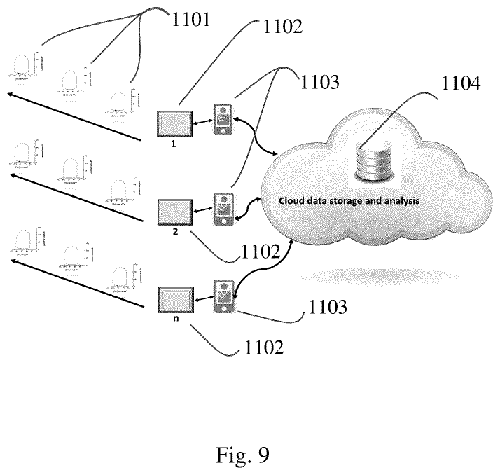

As shown in FIG. 9, the results of each examination (parameters derived from pressure-volume data) may be stored on the external device 1102 for comparison with previous or following examinations. To allow for comparisons involving more than one patient, the examination results may preferably be sent to cloud servers associated with the invention 1104, for example by means of smartphones or other personal devices 1103. These devices may communicate with the external device 1102 for example by means of Bluetooth or other communications means, while the personal device 1103 may communicate with the internet by means of Wi-Fi or other networking means. This will enable physicians and/or algorithms of the system to perform individual trend analysis 1101 to determine if the patient's cardiac health has remained stable, has deteriorated or improved, in addition to allowing for cohort analyses of various sorts involving more than one patient.

Each examination result may be also uploaded to a cloud database 1104 (FIG. 9) via the internet or any other communication mode using a smartphone, PC, etc. This will allow for the creation of a large population-based database which will enable users and/or algorithms of the invention to perform trend analyses for each individual patient as described above as well as compare each patient's results to constantly updated normal and pathological pressure-volume data parameter ranges, while more and more examination results of multiple patients are accumulated. This allows the implementation of artificial intelligence (AI) tools and machine learning algorithms to analyze the pressure-volume data, and recognize patterns in such data pertaining to single patients as well as cohorts of many patients, and make predictions or decisions based on that analysis.

The physician may be updated with examination results either directly from the external unit or through the cloud database and data processing system. Alerts may be issued when required allowing for appropriate pharmacological adjustments to be done accordingly.

Although as described in FIG. 2, the invention is intended to be used at the outpatient or home setting, the high quality, operator-independent measurements provided by the technology and based on the invention, may be used in the hospital or clinic setting where the technology could be integrated into existing or future ultrasound devices and transducers. This will provide clinicians with a reliable, consistent way for assessing the existence and severity of heart failure, especially in the subset of patients suffering from diastolic dysfunction or HFpEF.

### Data and Reporting

Each examination result may be also uploaded to a cloud database via the internet or any other communication mode using a smartphone, PC, etc. This will allow for the creation of a large population-based database which will enable to perform a trend analysis for each individual patient as described above as well as compare each patient's results to constantly updated normal and pathological pressure-volume data parameter ranges while more and more examination results of multiple patients are accumulated. This may implement artificial intelligence (AI) tools and algorithms to analyze the pressure-volume data, recognize patterns, and make predictions or decisions based on that analysis.

The cloud database (or any equivalent thereof) will be used to store and analyze individual and population data, not only of cardiac function parameters derived from analysis of pressure-volume loops but also any the other clinically significant parameters which will be derived from ventricular volume alone. These include among others Ejection Fraction, End-diastolic Volume, Stroke Volume, Cardiac Output, Cardiac Index and Global Longitudinal Shortening (GLS). Furthermore, the database will enable to correlate any parameter of cardiac function generated by the invention to all other clinically relevant information (i.e., age, gender, weight, blood pressure, medications, comorbidities, past clinical events and hospitalizations, etc.)

The physician may be updated with examination results either directly from the external unit or through the cloud database and data processing system. Alerts may be issued when required allowing for appropriate pharmacological adjustments to be done accordingly.

### General Remarks

Although the invention is primarily intended to be used by patients at home (after placement of the implanted elements of the invention) it may provide significant value also at hospital and Point of Care (POC) clinics. The current practice of echocardiography is often subject to interobserver variability in determining critical parameters such as Ejection Fraction (EF), due to measurement variability such as measurements by different operators being performed in slightly different planes. This issue is solved by the invention, as it relies on detection of the implanted sensors which always stay at the same location.

Another advantage of the invention over imaging ultrasound is that it does not rely on acquisition and analysis (by expert or AI) of high-resolution ultrasound images, a method which will have unavoidable inaccuracies due (for instance) to operator variability and/or inaccuracies due to estimation of a 3D volume from a 2D image. The passive ultrasound reflectors of the invention will generate a high SNR, and will thus be detectable with a high level of reliability and consistency.

The ability to generate and analyze pressure-volume loops of a cardiac chamber as described in the invention provides a reliable means for the monitoring of a large group of patients suffering from heart failure with a preserved ejection fraction (HFpEF) where measurements of volumes alone may not provide the clinically relevant information and proper assessment of the severity of the disease.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

1. A system for wireless cardiac diagnostics comprising:
a. a set of wireless position sensors deployed in a cardiac chamber;
b. wireless pressure sensing means adapted for sensing the pressure within said cardiac chamber;
c. an external device adapted to:
i. determine the displacements between said wireless position sensors;
ii. determine the pressure from said pressure sensing means;
iii. determine one or more pressure-volume loops based on said displacements and said pressure;
iv. determine instantaneous measures of cardiac health based on said pressure-volume loop;
whereby cardiac health is determined wirelessly, non-invasively, continuously, and without the need to acquire or analyze high quality echocardiographic images.

2. The system of claim 1 further providing a cloud server adapted to store and analyze said pressure volume loops, and communications means adapted to transfer information between said cloud server and said external device, said cloud server performing analyses selected from the group consisting of:
a. comparison of parameters derived from said pressure-volume loops with parameters derived from previous pressure-volume loops for a given patient so as to perform patient-specific trend analysis;
b. comparison of parameters derived from said pressure-volume loops with normal and pathological pressure-volume parameter ranges derived from a constantly updated database from multiple patients.
c. Performing correlations between parameters derived from said pressure volume loops and other clinical parameters relevant for the assessment of cardiac health.

3. The system of claim 1 wherein said wireless position sensors are solid, porous, or hollow forms selected from the group consisting of: sphere, coil, cylinder, polyhedron, corner cube, ellipsoid, ring, shapes adapted to have high reflectivity, and arrays of any of these shapes.

4. The system of claim 1 wherein said wireless position sensors are passive electromagnetic or ultrasound reflectors.

5. The system of claim 1 wherein further indicators of cardiac health selected from the group consisting of: Ejection Fraction, End-diastolic Volume, Stroke Volume, Cardiac Output, Cardiac Index, Global Longitudinal Shortening (GLS), and any function of ventricular function are measured.

6. The system of claim 1 wherein said wireless position sensors have one or more distinct resonances at particular frequencies, and wherein said external device uses electromagnetic or ultrasound transmitters and receivers to induce and sense said resonances of said wireless position sensors.

7. The system of claim 1 wherein said pressure sensor resonant frequencies are affected by the external pressure surrounding said sensors, whereby said pressure may be determined by correlating the measured value of said resonance frequency with a predetermined calibration curve relating external pressure to resonance frequency.

8. The system of claim 6 wherein said sensors are nonrigid and compressible.

9. The system of claim 1 wherein said external device uses true range multilateration or triangulation to determine said displacement of said wireless position sensors.

10. The system of claim 1 wherein said external device extracts and processes raw spatial data from said ultrasound transducer(s) to determine said displacement of said wireless position sensors.

11. The system of claim 1 wherein said pressure sensing means is wireless.

12. The system of claim 1 wherein said pressure sensing means is incorporated into one or more of said wireless position sensors.

13. The system of claim 1 wherein said pressure sensing means employs a passive sensor having a pressure-dependent resonant frequency.

14. The system of claim 1 wherein parameters derived from said pressure-volume loops from a given examination of a patient are used in comparison to parameters derived from previous pressure-volume loops measured from said patient to determine changes in said cardiac health over time.

15. A method for noninvasive determination of cardiac pressure-volume loops comprising the steps:
a. affixing two or more wireless position sensors and a wireless pressure sensor in a cardiac chamber;
b. continuously determining the displacement(s) between said position sensors by means of an external device adapted for this determination;
c. continuously determining the pressure in said cardiac volume by means of said external device;
d. forming pressure-volume loops from said displacement and pressure measurements;
e. calculating parameters of said pressure-volume loops adapted to indicate cardiac health;
whereby cardiac health is determined wirelessly, non-invasively, continuously, and without the need to acquire or analyze high-quality echocardiographic images.

16. The method of claim 15 further providing a cloud server adapted to store and analyze said pressure-volume loops, and communications means adapted to transfer information between said cloud server and said external device, said cloud server performing analyses selected from the group consisting of:
a. comparison of parameters derived from said pressure-volume loops with parameters derived from previous pressure-volume loops for a given patient so as to perform patient-specific trend analysis;
b. comparison of parameters derived from said pressure-volume loops with normal and pathological pressure-volume parameter ranges derived from a constantly updated database from multiple patients.
c. Performing correlations between parameters derived from said pressure volume loops and other clinical parameters relevant for the assessment of cardiac health.

17. The method of claim 15 wherein said wireless position sensors are solid, porous or hollow forms selected from the group consisting of: sphere, coil, cylinder, polyhedron, corner cube, ellipsoid, ring, shapes adapted to have distinct resonances, shapes adapted to have high reflectivity, and arrays of any of these shapes.

18. The method of claim 15 wherein said wireless position sensors are passive electromagnetic or ultrasound reflectors.

19. The method of claim 15 wherein said wireless position sensors have one or more distinct resonances at particular frequencies, and wherein said external device uses electromagnetic or ultrasound transmitters and receivers to induce and sense said resonances of said wireless position sensors.

20. The method of claim 15 wherein said frequencies are affected by the external pressure surrounding said sensors, whereby said pressure may be determined by correlating the measured value of said resonance frequency with a predetermined calibration curve relating external pressure to resonance frequency.

21. The method of claim 15 wherein said external device uses true range multilateration or triangulation to determine said displacement of said wireless position sensors.

22. The method of claim 15 wherein said external device extracts and processes raw spatial data from the ultrasound transducer(s) to determine said displacement of said wireless position sensors.

23. The method of claim 15 wherein said pressure sensing means is wireless.

24. The method of claim 15 wherein said pressure sensing means is incorporated into one or more of said wireless position sensors.

25. The method of claim 15 wherein said pressure sensing means employs a passive sensor having a pressure-dependent resonant frequency.

26. The method of claim 15 wherein parameters derived from said pressure-volume loops from a given examination of a patient are used in comparison to parameters derived from previous pressure-volume loops measured from said patient to determine changes in said cardiac health over time.

27. An implantable medical device for monitoring variable body geometry comprising:
a. one or more wireless position sensors deployed at a set of bodily positions;
b. an external device adapted to:
i. determine the displacements between said wireless position sensors;
ii. determine instantaneous measures of cardiac health based on said displacements;
whereby variable body geometry may be monitored continuously, noninvasively, and without the need to acquire or analyze high-quality echocardiographic images.
